(19) United States
(12) Patent Application Publication
(10) Pub. No.: US 2015/0148410 A1
(43) Pub. Date: May 28, 2015

(54) IDENTIFICATION OF SPECIFIC APOLIPOPROTEIN EPITOPES ON CIRCULATING ATHEROGENIC LOW-DENSITY LIPOPROTEIN

(71) Applicant: Texas Heart Institute, Houston, TX (US)

(72) Inventors: Chu-Huang Chen, Houston, TX (US); Liang-Yin Ke, Kaohsiung city (TW)

(73) Assignee: Texas Heart Institute, Houston, TX (US)

(21) Appl. No.: 14/343,529

(22) PCT Filed: Sep. 10, 2012
(86) PCT No.: PCT/US2012/054455

§ 371 Date: Aug. 12, 2014

Related U.S. Application Data
(60) Provisional application No. 61/532,920, filed on Sep. 9, 2011.

(51) Int. Cl. G01N 33/92; A61K 31/366; C07K 14/705; C07K 14/775

(52) U.S. Cl. CPC G01N 33/92; C07K 14/775; A61K 31/366; C07K 14/705; G01N 2333/775; G01N 2800/50; G01N 2800/324; G01N 2800/7019
USPC 514/460; 530/322; 530/359; 436/501; 435/7.92; 435/69.1

(57) ABSTRACT

An isolated peptidic fragment of apolipoprotein E comprises at least 3 contiguous amino acids, Including glycosylated threonine 194, threonine 289, serine 94, or serine 76 of SEQ ID NO.: 1, or any combination of those. An antibody capable of binding to the isolated peptidic fragment. A method of detecting a naturally-occurring circulating atherogenic low-density lipoprotein in a plasma sample from an individual, comprising qualitatively and/or quantitatively detecting in a low-density lipoprotein that binds to the antibody. A method of assessing an individual's risk of ischemic heart disease and/or atherosclerosis comprises quantifying in a plasma sample from the individual an amount of apolipoprotein E comprising glycosylated threonine 194, threonine 289, serine 94 or serine 76 of SEQ ID NO.: 1, or any combination of those.

### CROSS-REFERENCE TO RELATED APPLICATIONS

This application claims priority to Provisional application No. 61/532,920, filed on Sep. 9, 2011.

### TECHNICAL FIELD

This disclosure generally relates to the diagnosis and targeted treatment of ischemic heart disease and atherosclerosis, and more particularly to compositions and methods for identifying and quantifying specific epitopes on circulating atherogenic low-density lipoprotein (LDL), in the absence of artificial modification of LDL.

### BACKGROUND

Low-density lipoprotein (LDL) is normally metabolized by liver and vascular cells via the LDL receptor (LDLR). Homozygous or heterozygous genetic defects in LDLR expression lead to plasma LDL accumulation and premature coronary artery disease (CAD) as well as other atherosclerotic vascular abnormalities^{1, 2}. Elevation of plasma LDL cholesterol (LDL-C) alone may not be sufficient to induce vascular changes and it has been thought that LDL modification, such as oxidation, plays an important role in generating LDL's atherogenicity^{3, 4}. However, oxidized LDL equivalent to what has been produced experimentally in vitro has not been isolated from human plasma for mechanistic scrutiny. Evidence is now accumulating that LDL which carries a greater negative charge than the majority of LDL particles may be responsible for atherogenicity in dyslipidemia⁵⁻⁸. For simplicity, these highly negatively charged LDL particles have been called “electronegative” LDL, although in reality it is a relative term and should not be used to imply that the other LDL particles are “electropositive.”

Since the initial characterization of human atheroma-derived LDL by Hoff, Gotto, and associates in the USA, the term “electronegative LDL” has been used to describe LDL with fast relative electrophoretic mobility on agarose gel¹⁵⁻¹⁷. By use of fast protein liquid chromatography (FPLC) through ion-exchange columns, Avogaro and colleagues in Italy, divided human plasma LDL dichotomously into electropositive LDL(+) and electronegative LDL(−)¹⁸. Since then, several groups, in particular, the team led by Sanchez-Quesada in Spain, have described the chemical composition and functional characteristics of LDL(−), isolated by a similar protocol^{6, 19-33}.

Using a different protocol of anion-exchange chromatography, human plasma LDL obtained from patients with increased cardiac risks (hypercholesterolemia, type 2 diabetes mellitus, smoking) was divided into five (5) subtractions, L1 to L5, in order of increasing electronegativity, with L5 representing a pure and highly negatively charged LDL entity⁸⁻¹¹. L5, the most negatively charged, is the only subtraction that can induce endothelial dysfunction in cultivated arteries and atherogenic responses in cultured vascular cells. It also impairs normal differentiation of endothelial progenitor cells^{7, 8, 10-14}. Of importance, L5 is not recognized by LDLR, and blocking LDLR does not reduce L5's proapoptotic effects on vascular endothelial cells (ECs)⁷. In fact, L5 signals through, and is internalized by, lectin-like oxidized LDL receptor-1 (LOX-1) in both ECs and endothelial progenitor cells (EPCs)^{7, 8}.

Two other kinds of abnormal LDL, oxidized LDL and small-dense LDL, have received a great deal of attention and are being considered atherogenic by some. Unfortunately, neither oxidized LDL nor small-dense LDL has been isolated from human plasma or tissue to test their biological effects in cultured cells, therefore, no molecular evidence can be established. A critical issue in targeted treatment for ischemic heart disease and atherosclerosis in general is the identification of naturally-occurring circulating lipoprotein species capable of inducing atherogenic responses in vascular cells in the absence of artificial modification.

### SUMMARY

In accordance with certain embodiments, an isolated peptidic fragment of apolipoprotein comprises from 3 up to 298 contiguous amino acids of the translated region of apoE (SEQ ID NO.: 1), said fragment including at least one amino acid selected from the group consisting of threonine 194, threonine 289, serine 94, and serine 76 of SEQ ID NO.: 1, wherein at least one of the selected amino acids is glycosylated. Such peptidic fragments are sometimes also referred to herein as glycosylated polypeptides.

In some embodiments, at least one glycosylated amino acid is O-substituted with either N-acetylglucosamine-mannose-sialic acid or N-acetylglucosamine-mannose-mannose-mannose-sialic acid.

In some embodiments, an isolated peptidic fragment of claim 1 comprises 3-20 contiguous amino acids. For example, AATVGSLAGQPLQER (SEQ ID NO.: 2) wherein T is glycosylated, EQGRVRAATVGSLAGQPLQE (SEQ ID NO.: 3) wherein T is glycosylated, EKVQAAVGTSAAPVPSDN (SEQ ID NO.: 4) wherein T is glycosylated, VQAAVGTSAAPVPSDNH (SEQ ID NO.: 5) wherein T is glycosylated, EETRARLSKELQAAQAR (SEQ ID NO.: 6) wherein S is glycosylated, or LSKELQA (SEQ ID NO.: 7) wherein S is glycosylated.

Also provided in accordance with certain embodiments is a method of isolating an atherogenic LDL fraction (L5), which comprises loading isolated LDL onto an ion-exchange resin and eluting LDL subfractions from the resin step-wise according to the sequence: a) 0% B for 10 minutes, b) gradient from 0% to 15% B over the next 10 minutes, (c) gradient from 15% to 20% B over the next 30 minutes; d) isocratic at 20% B for 10 minutes, e) gradient from 20% to 100% B over the next 20 minutes, f) isocratic at 100% B for 10 minutes, and then g) gradient from 100% to 0% B over the next 5 minutes, and collecting five separate subfractions with increasing electronegativity, with L5 being the most negatively charged. In some embodiments, at least one gradient is a linear gradient. In some embodiments, one or more gradient is non-linear.

Also provided in accordance with certain embodiments is an antibody capable of selectively binding to any of the above-described peptidic fragments. In accordance with certain embodiments, a method of detecting a naturally-occurring circulating atherogenic low-density lipoprotein in a plasma sample from an individual is provided which comprises qualitatively and/or quantitatively detecting in the plasma sample a glycosylated apoliprotein E that selectively binds to an above-described antibody.

In accordance with still other embodiments, a method of detecting a naturally-occurring circulating atherogenic low-density lipoprotein in a plasma sample from an individual is provided. This method comprises qualitatively and/or quantitatively detecting in the plasma sample a glycosylated apolipoprotein E bearing N-acetylglucosamine-mannose-sialic acid or N-acetylglucosamine-mannose-mannose-mannose-sialic acid, or both.

A method of assessing an individual's risk of ischemic heart disease and/or atherosclerosis is provided in certain embodiments. This method comprises quantifying in a plasma sample from the individual an amount of apolipoprotein E comprising at least one glycosylated amino acid selected from the group consisting of glycosylated threonine 194, threonine 289, serine 94, and serine 76 of SEQ ID NO.: 1.

In some embodiments, a quantified amount of glycosylated apolipoprotein E exceeding 0.05% (wt/wt total LDL) indicates increased risk of ischemic heart disease and/or atherosclerosis. In some embodiments, quantifying glycosylated apolipoprotein E includes performing an immunoassay on a plasma sample, wherein the immunoassay utilizes an antibody capable of binding to an above-described glycosylated peptidic fragment. In some embodiments, an above-described method includes comparing a quantified amount of glycosylated apoliprotein E to a control value.

In accordance with certain embodiments, a method of screening a population of individuals for increased risk of ischemic heart disease and/or atherosclerosis is provided which comprises testing plasma samples from respective individuals for levels of apolipoprotein E comprising at least one glycosylated amino acid selected from the group consisting of glycosylated threonine 194, threonine 289, serine 94, and serine 76 of SEQ ID NO.: 1. The method further includes selecting the tested individuals having a level of said glycosylated apolipoprotein E that exceeds 0.05% (wt/wt total LDL); and treating at least the selected individuals with a therapeutic agent to decrease risk of ischemic heart disease and/or atherosclerosis. In some embodiments, the therapeutic agent is a lipid-lowering agent.

In accordance with still other embodiments, a method of cloning a selective receptor for an atherogenic low-density lipoprotein containing glycosylated residues on apoE is provided. This method includes obtaining a peptidic fragment of the translated region of apolipoprotein E (SEQ ID NO.: 1) comprising 3-20 contiguous amino acids including at least one amino acid selected from the group consisting of threonine 194, threonine 289, serine 94, and serine 76, wherein the selected amino acids are glycosylated; and then using the glycosylated peptidic fragment as a selective binding agent to induce synthesis of the receptor in a cellular expression system and/or using the glycosylated polypeptide for affinity purification of the receptor. In some embodiments, the glycosylated amino acids on the peptidic fragment are O-substituted with N-acetylglucosamine-mannose-sialic acid and/or N-acetylglucosamine-mannose-mannose-mannose-sialic acid. These and other embodiments, features and advantages will be apparent in the detailed description and drawings.

### BRIEF DESCRIPTION OF THE DRAWINGS

FIG. 1 is a box flow diagram illustrating a process for isolating an atherogenic L5 fraction of LDL, in accordance with an embodiment of the invention.

FIG. 2 shows the amino acid sequence of apoliprotein E including the untranslated 18 amino acid signal peptide preceding the translated 299 amino acid sequence (SEQ ID NO.: 1).

FIG. 3 shows the results of two-dimensional electrophoresis of unmodified and glycosylated apoE in L5, in which four apoE spots are separated (approximately 36 kDa), the lower two peaks are unglycosylated, the left upper peak is O-glycosylated on Thr194 and Thr289, (M/z+1312.4737). The right upper peak is O-glycosylated on Ser94 and Thr194. (M/z+2145.7703).

FIG. 4 shows that ApoE Thr194 O-Glycosylation pattern is determined by peptide molecular weight difference and glycol peptide mass calculation. Peptide AATVGSLAGQPLQER (aa 192-206, no signal peptide) showed four (4) different molecular weights: 1497.7983, 1700.8823, 1862.9412 and 2154.0366. Accordingly, the glycans on Thr194 are in the sequence of N-acetylglucosamine (M/z+203.084), mannose (M/z+162.0589) and sialic acid (M/z+291.0954).

FIG. 5 shows that ApoE Thr289 O-Glycosylation pattern is determined by peptide molecular weight difference and glycol peptide mass calculation. Peptide VQAAVGTSAAPVPSDNH (aa 283-299, no signal peptide) showed four (4) different molecular weights: 1620.7976, 1823.3727, 1985.955 and 2277.0369. Accordingly, the glycans on Thr289 are in the sequence of N-acetylglucosamine (M/z+203.0794), mannose (M/z+162.0528) and sialic acid (M/z+291.0954).

FIG. 6 O-glycosylation is schematically illustrated by N-acetylglucosamine (white squares), mannose (gray circles) and sialic acid (black diamonds). (a)=N-acetylglucosamine-mannose-sialic acid; (b)=N-acetylglucosamine-mannose-mannose-mannose-sialic acid.

### DEFINITIONS

In the following discussion and in the claims, the terms “comprising,” “including” and “containing” are used in an open-ended fashion, and thus should be interpreted to mean “including, but not limited to . . . ”.

The singular forms “a,” “an,” and “the” include plural referents unless the context clearly dictates otherwise.

The term “about,” when used in the context of a numerical value, means approximately or reasonably close to the given number, and generally includes, but is not limited to, ±10% of the stated number.

The term “and/or” includes “and” and, in the alternative, “or.”

The term “isolated peptidic fragment” refers to a synthetic, purified or partially purified peptide having an amino acid sequence matching that of a contiguous sequence of amino acids constituting a fragment or portion of a naturally-occurring larger amino acid sequence.

The term “polypeptide fragment” as used herein refers to a polypeptide that has an amino-terminal and/or carboxy-terminal deletion, wherein the remaining amino acid sequence is identical to the corresponding positions in the naturally-occurring sequence deduced, for example, from a full-length cDNA sequence. Fragments typically are at least about 5 to 14 amino acids long, and in some cases are at least about 20 amino acids long.

Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage such as —CH₂NH—, —CH₂S—, —CH₂—CH₂—, —CH═CH— (cis and trans), —COCH₂—, —CH(OH)CH₂—, and —CH₂SO—, by methods well known in the art.

The term “epitope” refers to any polypeptide determinant capable of selectively binding to an immunoglobulin or T-cell receptor. Epitopic determinants usually consist of chemically active surface groupings of molecules such as amino acids or sugar side chains and may, but not always, have specific three-dimensional structural characteristics, as well as specific charge characteristics. In general, an epitope is a region of an antigen that is selectively bound by an antibody. In certain cases, an epitope may include chemically active surface groupings of molecules such as amino acids, sugar side chains, phosphoryl, and/or sulfonyl groups. Additionally, an epitope may have specific three dimensional structural characteristics (e.g., a “conformational” epitope) and/or specific charge characteristics. An epitope is defined as “the same” as another epitope if a particular antibody selectively binds to both epitopes.

The term “increased risk” of ischemic heart disease or atherosclerosis refers to a greater likelihood of an individual's having existing ischemic heart disease or atherosclerosis, or of developing ischemic heart disease or atherosclerosis, compared to people who have L5 less than 0.5%.

The term “antibody” includes, without limitation, oligoclonal antibodies, monoclonal antibodies, polyclonal antibodies, dimers, multimers, multispecific antibodies (e.g., bispecific antibodies), chimeric antibodies, CDR-grafted antibodies, multi-specific antibodies, bi-specific antibodies, catalytic antibodies, chimeric antibodies, humanized antibodies, fully human antibodies, anti-idiotypic antibodies and antibodies that can be labeled in soluble or bound form as well as fragments, variants or derivatives thereof, either alone or in combination with other amino acid sequences provided by known techniques. An antibody may be from any species. An antibody is a protein generated by the immune system that is capable of recognizing and binding to a specific antigen (i.e., selectively binding the antigen). The term antibody also includes binding fragments of the antibodies of the invention; exemplary fragments include Fv, Fab, Fab′, single stranded antibody (svFC), dimeric variable region (Diabody) and disulphide stabilized variable region (dsFv). It has been shown that fragments of a whole antibody can perform the function of binding antigens. Examples of binding fragments are (1) the Fab fragment consisting of VL, VH, CL and CH1 domains¹, (2) the Fd fragment consisting of the VH and CH1 domains², (3) the Fv fragment consisting of the VL and VH domains of a single antibody³, (4) the dAb fragment, which consists of a VH or a VL domain; (5) isolated CDR regions, (6) F(ab′)₂ fragments, a bivalent fragment comprising two linked Fab fragments, (7) single chain Fv molecules (scFv), wherein a VH domain and a VL domain are linked by a peptide linker which allows the two domains to associate to form an antigen binding site⁴⁻⁵, (8) bispecific single chain Fv dimers (PCT/US92/09965) and (9) “diabodies”, multivalent or multispecific fragments constructed by gene fusion⁶. Fv, scFv or diabody molecules may be stabilized by the incorporation of disulphide bridges linking the VH and VL domains⁷. Minibodies comprising a scFv joined to a CH3 domain may also be made⁸. Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

“Fv,” when used herein, refers to the minimum fragment of an antibody that retains both antigen-recognition and antigen-binding sites.

“Fab,” when used herein, refers to a fragment of an antibody that comprises the constant domain of the light chain and the CH1 domain of the heavy chain.

“Label” or “labeled,” as used herein, refers to the addition of a detectable moiety to a polypeptide, for example, a radiolabel, fluorescent label, enzymatic label chemiluminescent labeled or a biotinyl group. Radioisotopes or radionuclides may include ³H, ¹⁴C, ¹⁵N, ³5S, ⁹⁰Y, ⁹⁹Tc, ¹¹¹In, ¹²⁵I, ¹³¹I, fluorescent labels may include rhodamine, lanthanide phosphors or FITC and enzymatic labels may include horseradish peroxidase, β-galactosidase, luciferase, alkaline phosphatase. Additional labels include, by way of illustration and not limitation: enzymes, such as glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase; dyes; additional fluorescent labels or fluorescers include, such as fluorescein and its derivatives, fluorochrome, GFP (GFP for “Green Fluorescent Protein”), dansyl, umbelliferone, phycoerythrin, phycocyanin, allophycocyanin, o-phthaldehyde, and fluorescamine; fluorophores such as lanthanide cryptates and chelates, e.g. Europium, etc.; chemoluminescent labels or chemiluminescers, such as isoluminol, luminol and the dioxetanes; sensitizers; coenzymes; enzyme substrates; particles, such as latex or carbon particles; metal sol; crystallite; liposomes; cells, etc., which may be further labelled with a dye, catalyst or other detectable group; molecules such as biotin, digoxygenin or 5-bromodeoxyuridine; toxin moieties, such as for example a toxin moiety selected from a group of Pseudomonas exotoxin (PE or a cytotoxic fragment or mutant thereof), Diptheria toxin or a cytotoxic fragment or mutant thereof, a botulinum toxin A, B, C, D, E or F, ricin or a cytotoxic fragment thereof, e.g., ricin A, abrin or a cytotoxic fragment thereof, saporin or a cytotoxic fragment thereof, pokeweed antiviral toxin or a cytotoxic fragment thereof and bryodin 1 or a cytotoxic fragment thereof.

The term “treatment” refers to preventing, deterring the occurrence of the disease or disorder, arresting, regressing, or providing relief from symptoms or side effects of the disease or disorder and/or prolonging the survival of the subject being treated.

The term “therapeutically effective amount” refers to that amount of the compound being administered that will relieve at least to some extent one or more of the symptoms of the disorder being treated. For example, an amount of the compound effective to prevent, alleviate or ameliorate symptoms of disease or prolong the survival of the subject being treated.

### DETAILED DESCRIPTION

At this time, negatively charged LDL, in particular L5, is the only naturally-occurring LDL that exhibits atherogenic properties without artificial modification. In healthy subjects, the most abundant and least negatively charged subfraction, L1, represents more than 98%, whereas L5 accounts for less than 0.5%, by weight of total LDL. In asymptomatic patients with cardiac risks, L5 may increase to 3-5% by weight of the total LDL, while in patients presenting with acute myocardial infarction, the percentage can increase to as high as 8-10%. Based on these findings, the inventors hypothesize that L5 is a circulating LDL entity responsible for atherothrombotic changes.

Sample Preparation.

Whole blood removed from hyperlipidemic (LDL-C >160 mg/dL) adult subjects with the approval of the internal review board at Baylor College of Medicine, Houston, Tex., USA, was protected by 1% penicillin/streptomycin and citrate phosphate dextrose adenine-1 from bacterial contamination and coagulation. The plasma obtained was treated with Complete Protease Inhibitor Cocktail (Roche; Cat. No. 05056489001; 1 tablet/100 mL) to prevent protein degradation. LDL (d=1.019-1.063 g/mL) was then isolated by sequential potassium bromide density centrifugation and treated with 5 mM EDTA and nitrogen to avoid ex vivo oxidation.

LDL Fractionation.

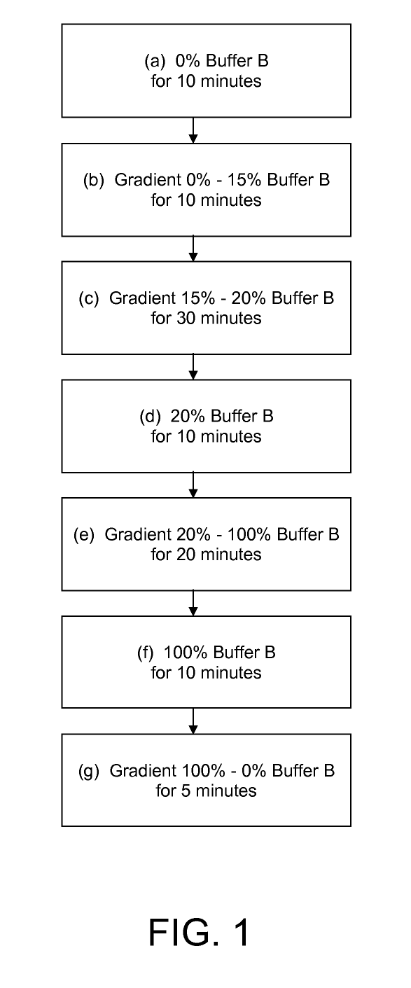

The LDL fractions were separated on UnoQ12 columns (BioRad) using 2 P-500 pumps controlled by an LCC-500 programmer. The columns were preequilibrated with buffer A (0.02 mol/L Tris HCl, pH 8.0, 0.5 mmol/L EDTA) in a 4° C. cold room. The EDTA-containing Tris HCl buffer used for chromatography was degassed. After dialysis with buffer A, up to 100 mg of LDL in 10 mL (10 mg/mL) was loaded onto the UnoQ12 column and eluted with a flow rate at 2 mL/min with a multistep gradient of buffer B (1 mol/L NaCl in buffer A). Elution was monitored at 280 nm with 2 AUFS. The gradient profile was run step-wise according to the following sequence: a) 0% B for 10 minutes, b) linear gradient from 0% to 15% B over the next 10 minutes, (c) linear gradient from 15% to 20% B over the next 30 minutes; d) isocratic at 20% B for 10 minutes, e) linear gradient from 20% to 100% B over the next 20 minutes, f) isocratic at 100% B for 10 minutes, and then g) linear gradient from 100% to 0% B over the next 5 minutes, as illustrated in FIG. 1. This gradient protocol, with a step-wise increased salt concentration performed in multiple steps, resolved LDL into 5 separate subfractions with increasing electronegativity, with L5 being the most negatively charged. These five fractions were collected, as indicated.

2-Dimensional Electrophoresis.

Protein contents of LDL subfractions were analyzed by 2-dimensional electrophoresis. Twice delipidated (1:1 EtAc+EtOH, 0.3 mL/30 μg LDL protein) L1 and L5 particles were centrifuged for 30 min (14000 rpm, 4° C.). After removal of the solution, the lipoprotein pellet was resuspended in 30 μL H₂O. Samples were incubated in ZOOM IPGRunner Cassette with Strip, 1× ZOOM 2D Protein Solubilizer 1, 1× Protease Inhibitor Cocktail, 20 mM DTT, and 3.5 mM Tris base at pH 7.4 for 2 hours. Two-dimensional-PAGE (isoelectrofocusing, equilibrating, performing) was performed by ZOOM IPGRunner, ZOOM Equilibration Tray and XCell SureLock Mini-Cel according to the user manual. The 4-20% 2-dimensional gels were then stained with SYPRO Ruby Protein Gel Stain (Ex/Em: 280, 450/610 nm).

LC/MS^{E} Analysis and Unique Protein Composition of L5.

Quantitative analysis was performed on a Waters Synapt HDMS mass spectrometer (Waters Corporation, MA, USA)⁵³⁻⁵⁵. In brief, total proteins isolated from each LDL subfraction were first digested with trypsin, and the resulting tryptic peptides were chromatographically separated on a Nano-Acquity separations module (Waters Corporation, MA, USA) incorporating a 50 fmol-on-column tryptic digest of yeast alcohol dehydrogenase as the internally spiked protein quantification standard. Peptide elution was executed through a 75 μm×25 cm BEH C-18 column under gradient conditions at a flow rate of 300 nL/min over 30 min at 35° C. The mobile phase was composed of acetonitrile as the organic modifier and formic acid (0.1% v/v) for molecule protonation. Mass spectrometry was performed on a Synapt HDMS instrument equipped with a nano-electrospray ionization interface and operated in the data-independent collection mode (MS^{E}). Parallel ion fragmentation was programmed to switch between low (4 eV) and high (15-45 eV) energies in the collision cell, and data were collected from 50 to 2000 m/z utilizing glu-fibrinopeptide B as the separate data channel lock mass calibrant. Data were processed with ProteinLynx GlobalServer v2.4 (Waters)⁵⁶. Deisotoped results were searched for protein association from the Uniprot human protein database (v15.12; containing 34,786 entries).

Characterization of Specific Antigenic Epitopes on the Surface of the L5 Particle.

An LDL particle is spherical and comprises an apolipoprotein frame containing neutral lipids (triglycerides, cholesteryl esters) in the core and other lipids (phospholipids, free cholesterol) on the surface. SDS-PAGE and two-dimensional electrophoresis (2DE) showed that the protein frame of L1 is composed mainly of apolipoprotein (apo) B-100, with an isoelectric point (pI) of 6.620. The protein composition of the LDL particle changes as the chromatographic subfractions become more electronegative. The more electronegative subfractions have increased levels of additional proteins in the LDL particle, including apoE (pI 5.5, apoA-I (pI 5.4), apoC-III (pI 5.1), and lipoprotein-a [Lp(a)] (pI 5.5), and a concomitant decrease in overall mole abundance of ApoB-100.

Because the proportional increases in the low-pI proteins may contribute to the negative charge of L5, we quantified the distribution of protein abundance in L1-L5 by using LC/MS^{E53-55}. On the basis of weight percentages (n=6), L1 contained 99% apoB-100 and trace amounts of other proteins. In contrast, L5 contained 60% apoB-100 and substantially increased amounts of Lp(a), apoE, apoA-1, apoCIII.

Glycosylation of apoE as a Specific Marker of L5.

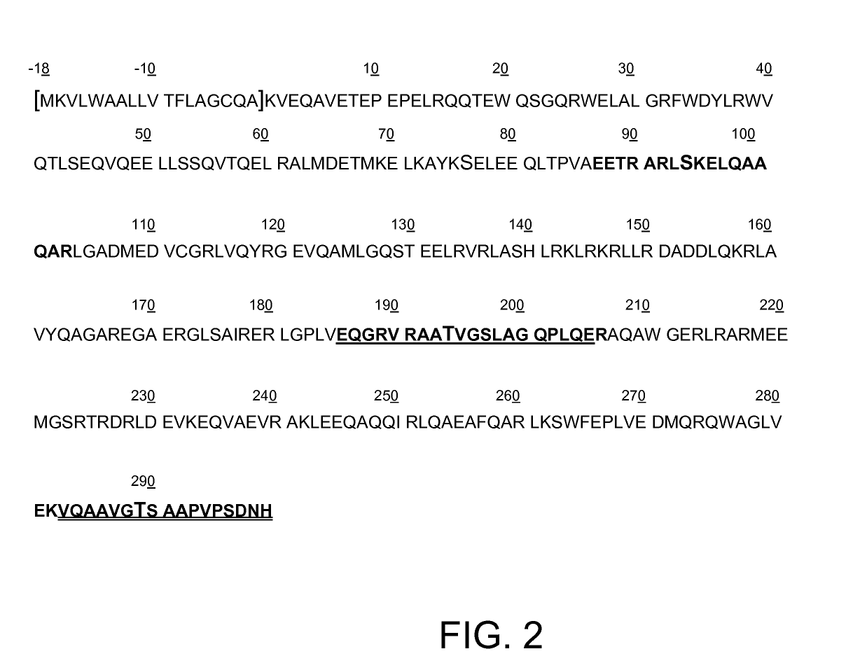

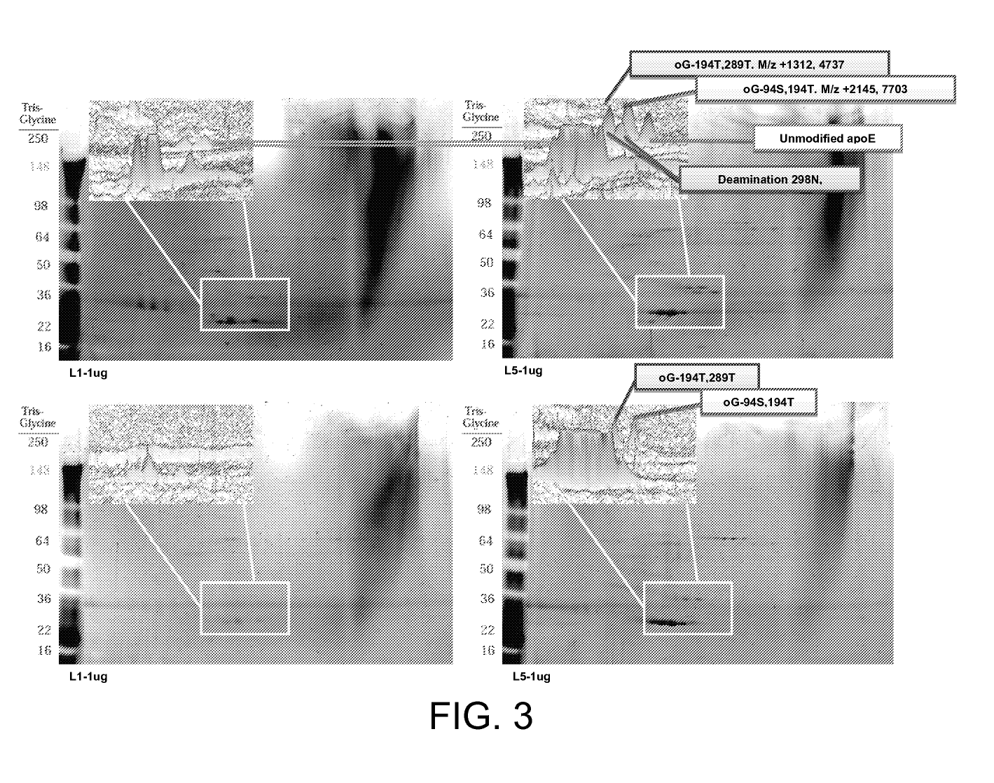

After careful examination, we came to the conclusion that the most unique posttranslational change that consistently and exclusively occurs in L5 is glycosylation of apoE. Using two-dimensional electrophoresis (2DE), four apoE spots were separated (approximately 36 kDa), as shown in FIG. 3. The underlying causes for the existence of these unique apoE spots include isoform mixtures (apoE2, E3, E4, in any combination), alternative splicing (SP1, SP2, SP3, SP4), and shorter transcripts (216-288 amino acids). By use of LC/MS analysis after human trypsin digestion of the excised gel spots, we determined that all these spots uniformly belong to the 299 amino acid translated portion (SEQ ID NO.: 1) of a 317 amino acid transcript shown in FIG. 2, and that the unique spots are a result of glycosylation instead of E2 or E4 mutation. Other forms of posttranslational modification, phosphorylation, deamidation, methionine oxidation, carbarnido-methylation, acetylation and N-terminal carbamylation, are excluded. The procedures used included conventional amino acid sequencing and detection of peptide molecular weight changes.

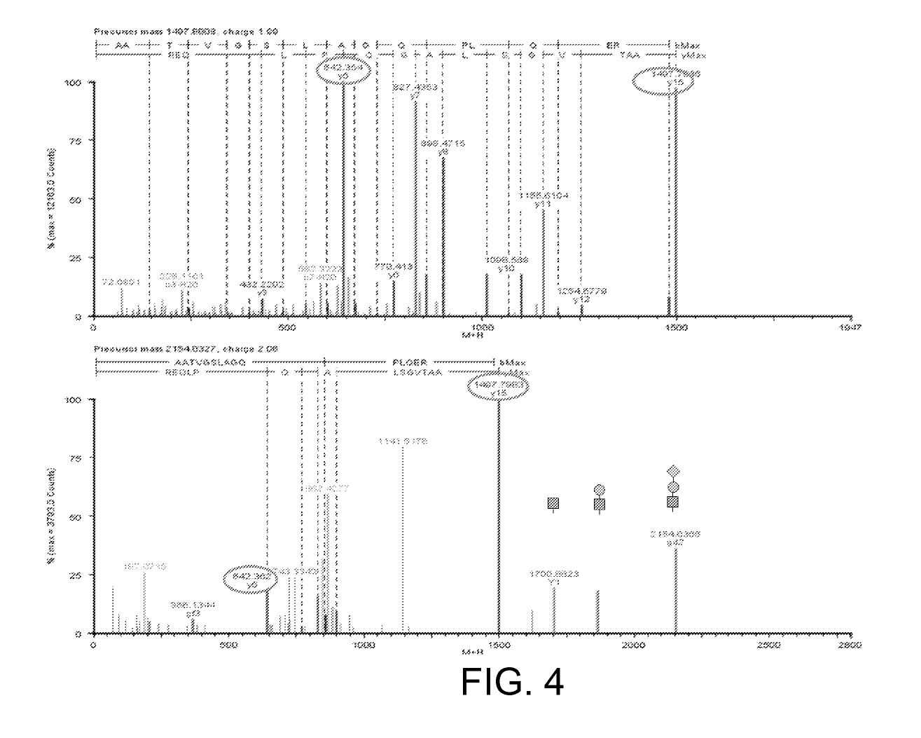

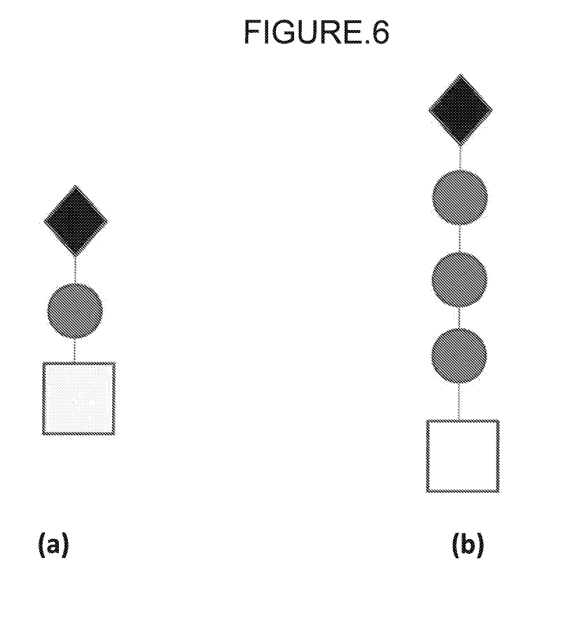

Two glycosylated apoE spots are consistently and exclusively detected in L5 preparations. No such glycosylated apoE is found in subfractions L1-L4. As revealed by 2DE in FIG. 3, the left upper left peak represents O-glycosylated on Thr194 and Thr289, (M/z+13 12.4737). The right upper peak depicts O-glycosylated on Ser94 and Thr194, (M/z+2145.7703). The lower two peaks are unglycosylated. These changes on apoE remain clearly identifiable in the unprocessed (without 2DE) L5 particles, with or without delipidation. The glycosylation pattern is determined by peptide molecular weight difference and glycol peptide mass calculation. O-glycosylation with N-acetylglucosamine, mannose and sialic acid stable sequence on threonine is consistently detected by LC/MS. For example, peptide AATVGSLAGQPLQER (SEQ ID NO.: 2), corresponding to aa 192-206 (no signal peptide) of the apoE transcript shown in FIG. 2, showed four different molecular weights: 1497.7983, 1700.8823, 1862.9412 and 2154.0366 (FIG. 4). Therefore, the glycans on Thr194 are N-acetylglucosamine (M/z+203.084), mannose (M/z+162.0589) and sialic acid (M/z+291.0954) in sequence, as schematically illustrated in FIG. 6a.

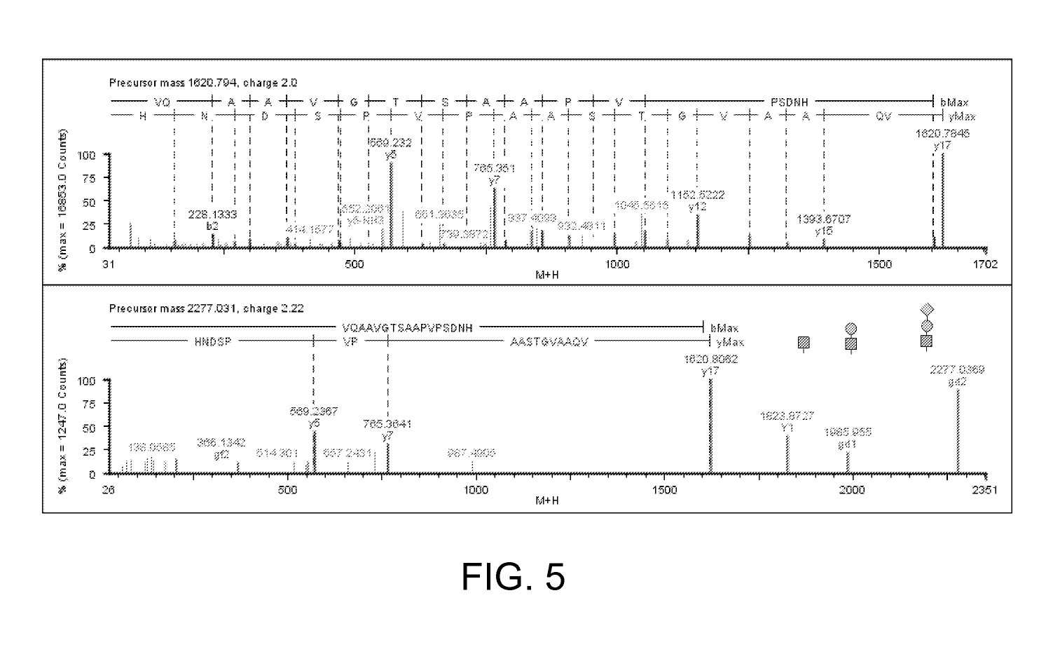

Peptide VQAAVGTSAAPVPSDNH (SEQ ID NO.: 4), corresponding to aa 283-299 (no signal peptide) of the apoE transcript shown in FIG. 2, showed molecular weights of 1620.7976, 1823.8727, 1985.955 and 2277.0369 (FIG. 5). Thus, the glycans on Thr289 represent N-acetylglucosamine (M/z+203.0794), mannose (M/z+1 62.0528) and sialic acid (M/z+291.0954) in sequence, as schematically illustrated in FIG. 6a. We also detected glycosylation on Ser94, which, in sequence, is represented by N-acetylglucosamine (M/z+203.0794), three (3) identical molecules of mannose (M/z+162.0528), and sialic acid (M/z+291.0954) (FIG. 6b).

On the basis of these findings, we have identified in apoE of L5 three (3) specific antigenic epitopes with a length of 17-20 amino acids:

EQGRVRAATVGSLAGQPLQE (SEQ ID NO.: 3), for glycosylated Thr194,

EKVQAAVGTSAAPVPSDN (SEQ ID NO.: 5), for glycosylated Thr289, and

EETRARLSKELQAAQAR (SEQ ID NO.: 6), for glycosylated Ser94, which can be seen in FIG. 2.

The glycans on Thr289 and on Ser 94 are schematically summarized in FIG. 6a, and the glycans on Thr194 are schematically summarized in FIG. 6b. Glycosylation on other L5 apoE sites, such as Ser76, may also occur, although less frequently than Thr194, Thr289 and Ser94. We propose that glycosylated apoE protein, and any peptidic fragment which spans one or more glycosylation sites in that protein (e.g., Thr194 and Thr289), are also potential antigenic epitopes that will function similarly to those specifically set forth herein. The sequences of all such fragments containing one or more glycosylated Thr194, Thr289 and/or Ser94 can be readily seen in FIG. 2 in the 299 aa sequence of the apoE transcript that is translated. In FIG. 2, the untranslated signal sequence is enclosed in brackets.

Production of ApoE Epitopes.

Glycosyl moieties on Threonine are L5-specific epitopes. For example, EQGRVRAATVGSLAGQPLQE (M/z 2395.56) and EKVQAAVGTSAAPVPSDN (M/z 2047.14) have N-acetylglucosamine+mannose+sialic acid glycan residue (M/z 656.59) on Threonine. A peptidic fragment of L5 apoE that can be used for antibody production contains at least three contiguous amino acids including at least one of the glycosylated amino acids Thr194, Thr289, Ser94 and Ser76. An immunogenic fragment may contain as few as 3 contiguous amino acids containing one of these glycosylated threonine or serine, up to, but not including the full 1-299 translated region of apoE. In some cases, the immunogenic peptidic fragments are approximately 15-20 amino acid long sequences. For example, AATVGSLAGQPLQER (SEQ ID NO.: 2) or EQGRVRAATVGSLAGQPLQE (SEQ ID NO.: 3) in which Thr194 is glycosylated; and EKVQAAVGTSAAPVPSDN (SEQ ID NO.: 4) and VQAAVGTSAAPVPSDNH (SEQ ID NO.: 5) in which Thr289 is glycosylated. Still other specific examples are EETRARLSKELQAAQAR (SEQ ID NO.: 6) or LSKELQA (SEQ ID NO.: 7), in which Ser94 is glycosylated. The amino acid sequences of many peptidic fragments can be readily seen in the translated region (i.e., amino acids 1-299) of apoE shown in FIG. 2.

The foregoing description demonstrates that glycosylated L5-specific epitopes can be isolated by fragmenting apo-E and using ultra-pure liquid chromatography (UPLC). Alternatively, the L5-specific glycosylated apo-E peptide sequences may be chemically synthesized and modified using known peptide synthetic methods and glycosylation techniques. For example, a 2D UPLC with the XBridge BEH130 C18 column (3.5 μm, 4.6×250 mm) may be used to isolate glycosylated apoEs (whole protein) based on their molecular weight and pH values. Alternatively, Glycan Separation Technology (GST) columns consisting of Waters hybrid-silica BEH Technology particles may be used to isolate glycol-peptide fragments.

Specific Antibodies to Specific Apoliprotein Epitopes on L5 apoE.

A disclosed glycosylated apoE peptide may be used for animal immunization and antibody purification. The glycosylated apoE peptides used to induce antibody formation contain from 3 contiguous amino acids up to the entire apoE sequence, excluding the untranslated 18 amino acid signal peptide preceding the translated 299 amino acid sequence (FIG. 2). For example, in some cases the peptides designated for antigens are 10-20 amino acids long. Antibodies may form against the particular 10-20 amino acid segment, or form against a segment up to the entire apoE, containing one or more glycol residues. In some cases, the glycosylated apoE peptides are coupled to a carrier protein or polypeptide, such as bovine serum albumin (BSA), to enhance antibody formation to short peptides of fewer than about 20 contiguous amino acids. A glycosylated apoE peptide may be covalently joined to BSA using known techniques for conjugating carrier proteins to peptides.

One method for generating fully human antibodies is through the use of XenoMouse® strains mice (Amgen, Inc., Fremont, Calif.) that have been engineered to contain up to, but less than, 1000 kb-sized germline configured fragments of the human heavy chain locus and kappa light chain locus⁵⁷. The Minilocus approach is an alternative approach. Exogenous Ig locus is mimicked through the inclusion of pieces (individual genes) from the Ig locus. Thus, one or more V_{H} genes, one or more D_{H} genes, one or more J_{H} genes, a mu constant region, and usually a second constant region (preferably a gamma constant region) are formed into a construct for insertion into an animal. This approach is described in U.S. Pat. No. 5,545,807 to Surani et al., and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay.

Once the antibody is expressed, the peptide ligand domain-containing polypeptides can be purified by traditional purification methods such as ionic exchange, size exclusion, or C18 chromatography. Protein detection and quantification of peptide ligand domain-containing polypeptides include silver staining, the BCA assay⁵⁸, the Lowry protein assay⁵⁹, and the Bradford protein assay⁶⁰.

For various applications, antibodies are obtained that are capable of selectively binding to a peptidic fragment of apoE that is glycosylated at one or more of Thr194, Thr289, Ser94 and Ser74. For example, any of SEQ ID NOs.: 2-6, or up to the full translated sequence of apoE (SEQ ID NO.: 1) may be used as the antigenic agent. An L5 apoE-specific monoclonal antibody may then be used as a diagnostic reagent or as a vaccine for clinical use, for example.

Identification of L5 Based on Glycosylated Epitopes on ApoE.

As demonstrated above, the presence of an atherogenic LDL subfraction (L5) in a plasma sample may be determined by direct chemical and physical analysis of a plasma LDL fraction to measure the amounts of one or more of glycosylated Thr194, Thr289, Ser94 and Ser74. Alternatively, L5 containing glycosylated apoE amino acid residues at one or more of Thr194, Thr289, Ser94 and Ser74 may be identified and quantified using any suitable immunologic technique. Examples of techniques that may be used to identify and quantify L5 epitopes include, but are not limited to, direct peptide sequencing by using mass spectrometry, indirect methods using various labeled antibody, such as Western blotting, enzyme-linked immunosorbent assay (ELISA), radio immunoassay (RIA), flow cytometry and any other antibody-based colorimetric assay. In one such immunologic method, a labeled monoclonal antibody specific for an isolated peptidic fragment of L5 apoE may be used in an immunoassay to detect circulating atherogenic L5 in a plasma sample from an individual.

Diagnosis Based on Detected Circulating Atherogenic L5.

To aid in determining an individual's relative level of risk of atherosclerosis and/or ischemic heart disease, a plasma sample obtained from the individual is assayed to detect at least one glycosylated amino acid selected from the group consisting of Thr194, Thr289, Ser94, and Ser76 of SEQ ID NO. 1. For example, an immunoassay may be performed on the plasma sample utilizing an antibody that specifically binds to a peptidic fragment described above. The detected amount of glycosylated amino acid is correlated to a level of L5 and compared to a control value. A control is obtained by measuring the corresponding level(s) in a pooled sample of healthy normolipemic individuals. For example, in some cases a detected amount of L5≧0.5% (wt/wt total LDL) indicates increased risk. In asymptomatic patients with cardiac risks, L5 may increase to 3-5% of the total LDL, while in patients presenting with acute myocardial infarction, the percentage can increase to as high as 8-10%. A healthcare provider may consider the results of this diagnostic test in making a determination as to therapeutic treatment of the individual, or the test may be utilized in assessing the success of a patient's existing risk-lowering therapeutic regime.

Screening Method to Aid in Treatment of Individuals at Increased Risk.

Individuals or groups of individuals at unknown risk for ischemic heart disease and/or atherosclerosis are screened by testing their plasma samples for levels of L5. Such testing includes measuring the amount of at least one glycosylated amino acid selected from the group consisting of Thr194, Thr289, Ser94, and Ser76 of SEQ ID NO.: 1. The individuals having an L5 level 0.05% (wt/wt total LDL) are identified, and at least those identified individuals with increased L5 levels are treated with a therapeutic agent, such as a lipid-lowering drug (e.g., statin) to decrease risk of ischemic heart disease and/or atherosclerosis. Patients receiving a lipid-lowering therapy may be periodically re-tested for L5 level as an aid to determining the effectiveness of the therapy.

Cloning an L5-specific receptor.

Negatively charged peptide sequences interfere with L5's binding affinity to the normal LDLR and apoER2. This forces L5 to be taken up by a receptor or receptors that have high affinities for negatively charged ligands. The inventors' preliminary studies suggest that L5, does not interact with the normal low density lipoprotein receptor (LDLR), but instead interacts with, and is internalized by, the positively charged lectin-like domain of LOX-1 receptor in both ECs and endothelial progenitor cells (EPCs), resulting in cell apoptosis^{7, 8}. The interaction between L5 and LOX-1 is believed to involve the above-described glycosylated residues on apoE. It is proposed that, beyond absolute LDL-C surplus in the plasma, the proportion of abnormal LDL (e.g., L5), that signals differently from normal LDL (e.g., L1), is an important factor for atherosclerosis. An L5-specific receptor that differs from the known LOX-1 receptor may be cloned using one or more of the disclosed glycosylated apoE peptides as binding agents to induce receptor synthesis and for affinity purification of the receptor, using known cloning techniques⁶¹⁻⁶². The modified apoE binding agent will contain the complete glyco-residues, including N-acetylglucosamine-mannose-sialic acid or N-acetylglucosamine-mannose-mannose-mannose-sialic acid. The terminal-end sialic acid residues make an essential contribution to the negative surface charge and hence the electrostastic attraction to specific targets. The peptides designed as binding agents to induce receptor synthesis or for affinity purification of the receptor comprise about 3-20 contiguous amino acids, such as any of SEQ ID NOs.: 2-7, for example. This size range differs from the range of glycosylated apoE peptides that may be used as antigens to induce antibody formation. Antigens suitable for inducing antibodies include glycosylated apoE peptides as large as SEQ ID NO.: 1. For some applications, the initial glycosylated apoE will be mutated or truncated to construct segmented peptides to enhance receptor localization capabilities. This will be followed by protein-protein interaction for receptor confirmation. The amino acid sequence of the isolated L5-specific receptor will then be derived using sequencing techniques that are known in the art, and the receptor will be used for development of targeted therapeutic agents for treatment of atherosclerosis and ischemic heart disease.

While the preferred embodiments have been shown and described, modifications thereof can be made by one skilled in the art without departing from the spirit and teachings of the invention. The embodiments described herein are exemplary and representative, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims. All patents, patent applications and publications cited herein are hereby incorporated herein by reference to the extent that they provide materials, methods and explanatory details supplementary to those set forth herein.

### REFERENCES

1. M. S. Brown, J. L. Goldstein. Science 185, 61-63 (1974).
2. J. L. Goldstein, M. S. Brown. Arterioscler Thromb Vasc Blot 29, 43 1-438 (2009).
3. J. L. Witztum, D. Steinberg. Trends Cardiovasc Med 11, 93-102 (2001).
4. P. Libby. Nature 420, 868-874 (2002).
5. C. Bancells, S. Benitez, J. Ordonez-Llanos, K. Oorni, P. T. Kovanen, R. W. Milne, J. L. Sanchez-Quesada. J Biol Chem 286, 1125-1133 (2011).
6. C. Bancells, S. Villegas, F. J. Blanco, S. Benitez, I. Gallego, L. Beloki, M. Perez-Cuellar, J. Ordonez-Llanos, J. L. Sanchez-Quesada. J Biol Chem 285, 32425-32435 (2010).
7. J. Lu, J. H. Yang, A. R. Burns, H. H. Chen, D. Tang, J. P. Walterscheid, S. Suzuki, C. Y. Yang, T. Sawamura, C. H. Chen. Circ Res 104, 619-627 (2009).
8. D. Tang, J. Lu, J. P. Walterscheid, H. H. Chen, D. A. Engler, T. Sawamura, P. Y. Chang, H. J. Safi, C. Y. Yang, C. H. Chen. J Lipid Res 49, 33-47 (2008).
9. C. H. Chen, T. Jiang, J. H. Yang, W. Jiang, J. Lu, G. K. Marathe, H. J. Pownall, C. M. Ballantyne, T. M. McIntyre, P. D. Henry, C. Y. Yang. Circulation 107, 2102-2108 (2003).
10. C. Y. Yang, J. L. Raya, H. H. Chen, C. H. Chen, Y. Abe, H. J. Pownall, A. A. Taylor, C. V. Smith. Arterioscler Thromb Vasc Biol 23, 1083-1090 (2003).
11. J. Lu, W. Jiang, J. H. Yang, P. Y. Chang, J. P. Walterscheid. H. H. Chen, M. Marcelli, D. Tang, Y. T. Lee, W. S. Liao, C. Y. Yang, C. H. Chen. Diabetes 57, 158-166 (2008).
12. C. Y. Yang, H. H. Chen, M. T. Huang, J. L. Raya, J. H. Yang, C. H. Chen, J. W. Gaubatz, H. J. Pownall, A. A. Taylor, C. M. Ballantyne, F. A. Jenniskens, C. V. Smith. Atherosclerosis 193, 283-291 (2007).
13. Y. Abe, M. Fornage, C. Y. Yang, N. A. Bui-Thanh, V. Wise, H. H. Chen, G. Rangaraj, C. M. Ballantyne. Atherosclerosis 192, 56-66 (2007).
14. H. H. Chen, B. D. Hosken, M. Huang, J. W. Gaubatz, C. L. Myers, R. D. Macfarfane, H. J. Pownall, C. Y. Yang. J Lipid Res 48, 177-184 (2007).
15. H. F. Hoff, M. Karagas, C. L. Heideman, J. W. Gaubatz, A. M. Gotto, Jr. Atherosclerosis 32, 259-268 (1979).
16. H. F. Hoff, J. W. Gaubatz. Atherosclerosis 42, 273-297 (1982).
17. B. A. Clevidence, R. E. Morton, G. West, D. M. Dusek, H. F. Hoff. Arteriosclerosis 4, 196-207 (1984).
18. P. Avogaro, G. B. Bon, G. Cazzolato. Arteriosclerosis 8, 79-87 (1988).
19. B. Chappey, I. Myara, M. O. Benoit, C. Maziere, J. C. Maziere, N. Moatti. Biochim Biophys Acta 1259, 261-270 (1995).
20. K. Demuth, I. Myara, B. Chappey, B. Vedie, M. A. Pech-Amsellem, M. E. Haberland, N. Moatti. Arterioscler Thromb Vasc Biol 16, 773-783 (1996).
21. J. L. Sanchez-Quesada, A. Perez, A. Caixas, J. Ordonmez-Llanos, G. Carreras, A. Payes, F. Gonzalez-Sastre, A. de Leiva. Diabetologia 39, 1469-1476 (1996).
22. A. Cordoba-Porras, J. L. Sanchez-Quesada, F. Gonzalez-Sastre, J. Ordonez-Lianas, F. Blanco-Vaca. J Mol Med 74, 771-776 (1996).
23. B. Vedie, X. Jeunemaitre, J. L. Megnien, I. Myara, H. Trebeden, A. Simon, N. Moatti. Arterioscler Thromb Vasc Biol 18, 1780-1789 (1998).
24. J. L. Sanchez-Quesada, C. Otal-Entraigas, M. Franco, O. Jorba, F. Gonzalez-Sastre, F. Blanco-Vaca. J. Ordonez-Llanos. Am J Cardiol 84, 655-659 (1999).
25. J. S. Fabjan, P. M. Abuja, R. J. Schaur, A. Sevanian. FEES Lett 499, 69-72 (2001).
26. S. Benitez, J. L. Sanchez-Quesada, V. Ribas, O. Jorba, F. Blanco-Vaca, F. Gonzalez-Sastre, J. Ordonez-Llanos. Circulation 108, 92-96 (2003).
27. O. Ziouzenkova, L. Asatryan, D. Sahady, G. Orasanu, S. Perrey, 6. Cutak, T. Hassell, T. E. Akiyama, J. P. Berger, A. Sevanian, J. Plutzky. J Biol Chem 278, 39874-39881 (2003).
28. J. L. Sanchez-Quesada, S. Benitez, J. Ordonez-Llanos. Curr Opin Lipidol 15, 329-335 (2004).
29. M. R. Barros, M. C. Bertolami, D. S. Abdalla, W. P. Ferreira. Atherosclerosis 184, 103-107 (2006).
30. J. A. Oliveira, A. Sevanian, R. J. Rodrigues, E. Apolinatio, D. S. Abdalla. Clin Biochem 39, 708-714 (2006).
31. C. Bancells, J. L. Sanchez-Quesada, R. Birkelund, J. Ordonez-Llanos, S. Benitez. J Lipid Res 51, 2947-2956 (2010).
32. R. Brunelli, G. Balogh, G. Costa, M. De Spirito, G. Greco, G. Mei, E. Nicolai, L. Vigh, F. Ursini, T. Parasassi. Biochemistry 49, 7297-7302 (2010).
33. C. Bancells, F. Canals, S. Benitez, N. Colome, J. Julve, J. Ordonez-Llanos, J. L. Sanchez-Quesada. J Lipid Res 51, 3508-3515 (2010).
34. M. Aviram. Biochem Med 30, 111-118 (1983).
35. A. Kawakami, M. Osaka, M. Tani, H. Azuma, F. M. Sacks, K. Shimokado, M. Yoshida. Circulation 118, 731-742 (2008).
36. K. Tziomalos, V. G. Athyros, A. S. Wierzbicki, D. P. Mikhailidis. Curr Opin Cardiol 24, 351-357 (2009).
37. J. C. Silva, M. V. Gorenstein, G. Z. Li, J. P. Vissers, S. J. Geromanos. Mol Cell Proteomics 5, 144-156 (2006).
38. J. C. Silva, R. Denny, C. A. Dorschel, M. Gorenstein, I. J. Kass, G. Z. Li, T. McKenna, M. J. Nold, K. Richardson, P. Young, S. Geromanos. Anal Chem 77, 2187-2200 (2005).
39. S. J. Geromanos, J. P. Vissers, J. C. Silva, C. A. Dorschel, G. Z. Li, M. V. Gorenstein, R. H. Bateman, J. I. Langridge. Proteomics 9, 1683-1695 (2009).
40. G. Z. Li, J. P. Vissers, J. C. Silva, D. Golick, M. V. Gorenstein, S. J. Geromanos. Proteomics 9, 1696-1719 (2009).
41. C. H. Chen. Curr Opin Lipidol 15, 337-341 (2004).
42. P. G. Scheffer, S. J. Bakker, R. J. Heine, T. Teerlink. Clin Chem 43, 1904-1912 (1997).
43. J. W. McLean, J. E. Tomlinson, W. J. Kuang, D. L. Eaton, E. Y. Chen, G. M. Fless, A. M. Scanu, R. M. Lawn. Nature 330, 132-137 (1987).
44. G. Uterrnann, H. J. Menzel, H. G. Kraft, H. C. Duba, H. G. Kemmler, C. Seitz. J Clin Invest 80, 458-465 (1987).
45. M. S. Brown, J. L. Goldstein. Science 232, 34-47 (1986).
46. C. Y. Yang, S. H. Chen, S. H. Gianturco, W. A. Bradley, J. T. Sparrow, M. Tanimura, W. H. Li, Q. A. Sparrow, H. DeLoof, M. Rosseneu, et al. Nature 323, 738-742 (1986).
47. A. Law, J. Scott. J Lipid Res 31, 1109-1120 (1990).
48. M. S. Brown, J. L. Goldstein. Proc Natl Acad Sci USA 76, 3330-3337 (1979).
49. K. H. Weisgraber, T. L. Innewrity, R. W. Mahley. J Biol Chem 253, 9053-9062 (1978).
50. U. P. Steinbrecher. J Biol Chem 262, 3603-3608 (1987).
51. F. J. Blanco, S. Villegas, S. Benitez, C. Bancells, T. Diercks, J. Ordonez-Llanos, J. L. Sanchez-Quesada. J Lipid Res 51, 1560-1 565 (2010).
52. T. Sawamura, N. Kurne, T. Aoyama, H. Moriwaki, H. Hoshikawa, Y. Aiba, T. Tanaka, S. Miwa, Y. Katsura, T. Kita, T. Masaki. Nature 386, 73-77 (1997).
53. Silva, J. C., et al. Quantitative proteomic analysis by accurate mass retention time pairs. Anal Chem 77, 2187-2200 (2005).
54. Silva, J. C., Gorenstein, M. V., Li, G. Z., Vissers, J. P. & Geromanos, S. J. Absolute quantification of proteins by LCMSE: a virtue of parallel MS acquisition. Mol Cell Proteomics 5, 144-156 (2006).
55. Geromanos, S. J., et al. The detection, correlation, and comparison of peptide precursor and product ions from data independent LC-MS with data dependant LC-MS/MS. Proteomics 9, 1683-1695 (2009).
56. Li, G. Z., et al. Database searching and accounting of multiplexed precursor and product ion spectra from the data independent analysis of simple and complex peptide mixtures. Proteomics 9, 1696-1719 (2009).
57. Mendez, M. J., et al. Functional transplant of megabase human immunoglobulin loci recapitulates human antibody response in mice. Nat Genet 15, 146-156 (1997).
58. Smith, P. K., et al. Measurement of protein using bicinchoninic acid. Anal Biochem 150, 76-85 (1985).
59. Lowry, O. H., Rosebrough, N. J., Farr, A. L. & Randall, R. J. Protein measurement with the Folin phenol reagent. J Biol Chem 193, 265-275 (1951).
60. Bradford, M. M. A rapid and sensitive method for the quantitation of microgram quantities of protein utilizing the principle of protein-dye binding. Anal Biochem 72, 248-254 (1976).
61. Stephan, Z. F. & Yurachek, E. C. Rapid fluorometric assay of LDL receptor activity by Dil-labeled LDL. J Lipid Res 34, 325-330 (1993).
62. Sawamura, T., et al. An endothelial receptor for oxidized low-density lipoprotein. Nature 386, 73-77 (1997).
63. Ward, E. S., Gussow, D., Griffiths, A. D., Jones, P. T. & Winter, G. Binding activities of a repertoire of single immunoglobulin variable domains secreted from Escherichia coli. Nature 341, 544-546 (1989).
64. McCafferty, J., Griffiths, A. D., Winter, G. & Chiswell, D. J. Phage antibodies: filamentous phage displaying antibody variable domains. Nature 348, 552-554 (1990).
65. Holt, L. J., Herring, C., Jespers, L. S., Woolven, B. P. & Tomlinson, I. M. Domain antibodies: proteins for therapy. Trends Biotechnol 21, 484-490 (2003).
66. Bird, R. E., et al. Single-chain antigen-binding proteins. Science 242, 423-426 (1988).
67. Huston, J. S., et al. Protein engineering of antibody binding sites: recovery of specific activity in an anti-digoxin single-chain Fv analogue produced in Escherichia coli. Proc Natl Acad Sci USA 85, 5879-5883 (1988).
68. Holliger, P., Prospero, T. & Winter, G. “Diabodies”: small bivalent and bispecific antibody fragments. Proc Natl Acad Sci USA 90, 6444-6448 (1993).
69. Reiter, Y., Brinkmann, U., Lee, B. & Pastan, I. Engineering antibody Fv fragments for cancer detection and therapy: disulfide-stabilized Fv fragments. Nat Biotechnol 14, 1239-1245 (1996).
70. Hu, S., et at Minibody: A novel engineered anti-carcinoembryonic antigen antibody fragment (single-chain Fv-CH3) which exhibits rapid, high-level targeting of xenografts. Cancer Res 56, 3055-3061 (1996).

What is claimed is:

1. An isolated peptidic fragment of apolipoprotein, comprising from 3 up to 298 contiguous amino acids of the translated region of apoE (SEQ ID NO.: 1), said fragment including at least one amino acid selected from the group consisting of threonine 194, threonine 289, serine 94, and serine 76, wherein at least one of the selected amino acids is glycosylated.

2. The isolated peptidic fragment of claim 1, wherein at least one said glycosylated amino acid is O-substituted with either N-acetylglucosamine-mannose-sialic acid or N-acetylglucosamine-mannose-mannose-mannose-sialic acid.

3. The isolated peptidic fragment of claim 1, wherein said fragment comprises 3-20 contiguous amino acids.

4. The isolated peptidic fragment of claim 1 wherein said fragment has the amino acid sequence AATVGSLAGQPLQER (SEQ ID NO.: 2) wherein T is glycosylated.

5. The isolated peptidic fragment of claim 1 wherein said fragment has the amino acid sequence EQGRVRAATVGSLAGQPLQE (SEQ ID NO.: 3) wherein T is glycosylated.

6. The isolated peptidic fragment of claim 1 wherein said fragment has the amino acid sequence EKVQAAVGTSAAPVPSDN (SEQ ID NO.: 4) wherein T is glycosylated.

7. The isolated peptidic fragment of claim 1 wherein said fragment has the amino acid sequence VQAAVGTSAAPVPSDNH (SEQ ID NO.: 5) wherein T is glycosylated.

8. The isolated peptidic fragment of claim 1 wherein said fragment has the amino acid sequence EETRARLSKELQAAQAR (SEQ ID NO.: 6) wherein S is glycosylated.

9. The isolated peptidic fragment of claim 1 wherein said fragment has the amino acid sequence LSKELQA (SEQ ID NO.: 7) wherein S is glycosylated.

10. The peptidic fragment of claim 1, wherein said fragment is bound to an antibody that is selective for said fragment.

11. A method of detecting a naturally-occurring circulating atherogenic low-density lipoprotein in a plasma sample from an individual, comprising:
qualitatively and/or quantitatively detecting in said sample: a glycosylated apolipoprotein E bearing N-acetylglucosamine-mannose-sialic acid; a glycosylated apolipoprotein E bearing N-acetylglucosamine-mannose-mannose-mannose-sialic acid; a glycosylated apolipoprotein E bearing both N-acetylglucosamine-mannose-sialic acid and N-acetylglucosamine-mannose-mannose-mannose-sialic acid; or a glycosylated apoliprotein E that selectively binds to the antibody of claim 10.

12. A method of assessing an individual's risk of ischemic heart disease and/or atherosclerosis, comprising:
quantifying in a plasma sample from the individual an amount of apolipoprotein E comprising at least one glycosylated amino acid selected from the group consisting of glycosylated threonine 194, threonine 289, serine 94, and serine 76 of SEQ ID NO.: 1; and
comparing the quantified amount of said apoliprotein E to a control value.

13. The method of claim 12, wherein a quantified amount of said glycosylated apoliprotein E exceeding 0.05% (wt/wt total LDL) indicates increased risk of ischemic heart disease and/or atherosclerosis.

14. The method of claim 12, wherein said quantifying comprises performing an immunoassay on the plasma sample, wherein the immunoassay utilizes an antibody capable of binding to the peptidic fragment of claim 1.

15. A method of screening a population of individuals for increased risk of ischemic heart disease and/or atherosclerosis, comprising:
testing plasma samples from respective individuals for levels of apolipoprotein E comprising at least one glycosylated amino acid selected from the group consisting of glycosylated threonine 194, threonine 289, serine 94, and serine 76 of SEQ ID NO.: 1;
selecting the tested individuals having a level of said glycosylated apolipoprotein E that exceeds 0.05% (wt/wt total LDL); and
treating at least the selected individuals with a therapeutic agent to decrease risk of ischemic heart disease and/or atherosclerosis.

16. The method of claim 15 wherein said therapeutic agent is a lipid-lowering agent.

17. A method of cloning a selective receptor for an atherogenic low-density lipoprotein containing glycosylated residues on apoE, comprising:
obtaining a peptidic fragment of the translated region of apolipoprotein E comprising 3-20 contiguous amino acids including at least one amino acid selected from the group consisting of threonine 194, threonine 289, serine 94, and serine 76 of SEQ ID NO.: 1, wherein at least one of the selected amino acids are glycosylated.

18. The method of claim 17 further comprising using said peptidic fragment as a selective binding agent to induce synthesis of said receptor in a cellular expression system.

19. The method of claim 17 further comprising using said peptidic fragment as a selective binding agent for affinity purification of said receptor.

20. The method of claim 17, wherein the glycosylated amino acids are O-substituted with N-acetylglucosamine-mannose-sialic acid and/or N-acetylglucosamine-mannose-mannose-mannose-sialic acid.


SEQUENCE LISTING (7 sequences)

SEQ ID NO: 1 - protein, 299 aa, Homo sapien
  apolipoprotein E
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 2 - protein, 15 aa, Homo sapien
  apolipoprotein E
AATVGSLAGQPLQER

SEQ ID NO: 3 - protein, 20 aa, Homo sapien
  apolipoprotein E
EQGRVRAATVGSLAGQPLQE

SEQ ID NO: 4 - protein, 18 aa, Homo sapien
  apolipoprotein E
EKVQAAVGTSAAPVPSDN

SEQ ID NO: 5 - protein, 17 aa, Homo sapien
  apolipoprotein E
VQAAVGTSAAPVPSDNH

SEQ ID NO: 6 - protein, 17 aa, Homo sapien
  apolipoprotein E
EETRARLSKELQAAQAR

SEQ ID NO: 7 - protein, 7 aa, Homo sapien
  apolipoprotein E
LSKELQA